(19) United States
(12) Patent Application Publication
(10) Pub. No.: US 2022/0150400 A1
(43) Pub. Date: May 12, 2022

(54) DUAL IMAGING DEVICE MONITORING APPARATUS AND METHODS

(71) Applicant: Binatone Electronics International Limited, Sheung Wan (CN)

(72) Inventor: Marco KHOO, Sheung Wan (CN)

(21) Appl. No.: 17/434,374

(22) PCT Filed: Oct. 14, 2020
(86) PCT No.: PCT/IB2020/059666

§ 371 Date: Aug. 26, 2021

(30) Foreign Application Priority Data
Oct. 14, 2019 (GB) 1914855.0

(51) Int. Cl. H04N 5/232; H04N 7/18; H04N 5/247; G06T 7/70; G06V 40/10; G06V 40/16; A61B 5/08; A61B 5/11; A61B 5/16

(52) U.S. Cl. CPC H04N 5/23219; A61B 2503/04; H04N 5/247; G06T 7/70; H04N 5/23229; H04N 5/23299; G06V 40/103; G06V 40/161; G06V 40/174; A61B 5/0816; A61B 5/1118; A61B 5/165; G06V 2201/07; G06T 2207/10016; G06T 2207/30201; G06T 2207/30232; H04N 7/181

(57) ABSTRACT

A method of monitoring a target object uses a first imaging device to provide first images of a first area at a first resolution and processing the first images to detect a position of the target object in the first area. A second imaging device is used to provide second images of a second area that is a part of the first area and contains the detected position of the target object. The second images are at a second resolution that is higher than the first resolution. The second images may be analysed by a processor to determine characteristics of the target object.

### FIELD OF THE INVENTION

The invention relates to monitoring apparatus and methods of monitoring making use of dual imaging devices. Monitoring apparatus according to the invention is particularly suited to monitoring infants.

### BACKGROUND TO THE INVENTION

Known methods of infant monitoring using video require very clear and high-resolution imagery for processing unit calculation. This requires images with high pixel counts. Analysing high pixel count images covering relatively large areas requires the processing of very large amounts of data. Also, there are hardware limitations in terms of the image sensor and camera lens capacity required to obtain high clarity images for large areas.

### SUMMARY OF THE INVENTION

The invention provides a dual imaging device monitoring apparatus as specified in claim 1.

The invention also includes a method of monitoring a target object as specified in claim 9.

### BRIEF DESCRIPTION OF THE DRAWINGS

FIG. 1 is a schematic illustration of an infant monitoring apparatus according to the invention; and

FIG. 2 is a schematic representation of images captured by the video cameras of the infant monitoring apparatus; and

FIG. 3 illustrates a method of monitoring an infant using the infant monitoring apparatus.

### DETAILED DESCRIPTION

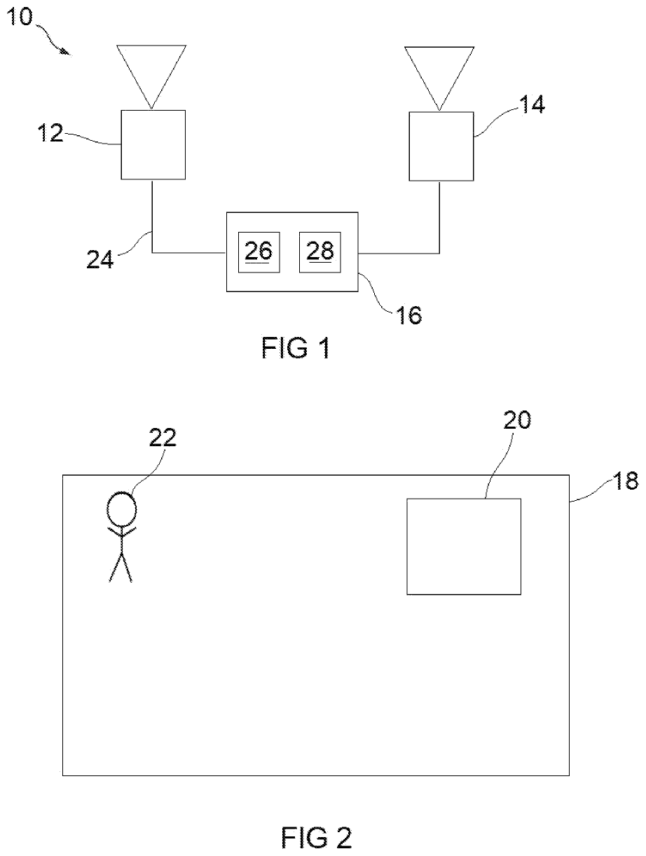

FIGS. 1 and 2 illustrate a dual imaging device monitoring apparatus in the form of an infant monitoring apparatus 10. The infant monitoring apparatus 10 comprises a first video camera 12, a second video camera 14 and a controller 16. The first video camera 12 is configured to capture images of a first area 18 and the second video camera 14 is configured capture images of a second area 20 that is within the first area. The controller 16 is connected with the first and second video cameras 12, 14 and configured to analyse images received from the first video camera 12 to detect the position of an infant 22 in the images received from the first video camera, control the second video camera 14 so that the second area 20 includes the detected position of the infant 22 and analyse the images from the second video camera 14 to determine the status of at least one predetermined infant characteristic.

The controller 16 may be connected with the first and second video cameras 12, 14 by a hard-wired connection 24. Alternatively, the controller 16 and the first and second video cameras 12, 14 may be wirelessly connected so that the controller receives image data from the first and second video cameras wirelessly and is also able to send commands wirelessly to at least the second video camera 14. The wireless communication may be over the internet via WIFI or by relatively shortrange radio frequency transmissions using, for example, a Bluetooth® protocol. In either case, the controller 16 may be located in an entirely separate room to the first and second video cameras 12, 14.

At least the second video camera 14 is configured to be controllable to adjust at least one of pan and tilt. The controller 16 is configured to control at least one of the pan and tilt of the second video camera 14 so that the second area 20 includes the detected position of the infant 22. The second video camera 14 may be fitted with suitable means for moving the camera to adjust the pan, tilt or both. Thus, the second video camera 14 may be provided with a traversing mechanism by which the camera can be caused to tilt, pan or both. The traversing mechanism may, for example, comprise at least one stepper motor, at least one servo motor or a combination thereof.

The first and second video cameras may have the same specification and may, for example, both be standard 720p video cameras.

The controller 16 may be a dedicated device having a processor and memory, an i/o system for receiving data from the first and second video cameras 12, 14, input commands from a user and outputting commands to at least the second video camera 14. The controller 16 may also be connected with a display device. Alternatively, the controller 16 may be configured as software that may run on a computer or microprocessor linked to a user interface that may comprise a keyboard or keypad and a display, and connected with the first and second video cameras 12, 14 via a hardwired network, WIFI or a radio frequency communications system using, for example, a Bluetooth® protocol. In other examples, a controller 16 configured as software may be distributed across multiple devices. For example, a computer or microprocessor may be configured to process images received from the first and second video cameras and output control signals to a separate controller that is configured to convert commands received from the computer or microprocessor and provide drive signals or other commands to the first and second video cameras 12, 14. In some examples, a part of the controller 16 function may be performed by software provided on cloud-based processors.

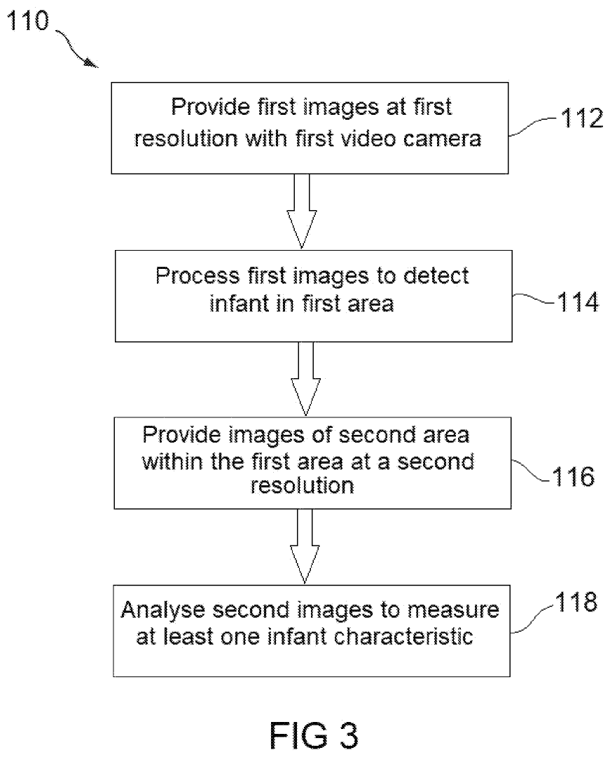

The infant monitoring apparatus 10 may be used to monitor a baby in a crib or an infant in a defined play area. For example, as shown in FIG. 3, a method of monitoring an infant 110 may comprise the step 112 of using the first video camera 12 to provide first images of the first area 18 at a first resolution. The method may additionally comprise the step 114 of processing the first images to detect a position of the infant 22 in the first area 18. The position of the infant 22 may be detected by running at least one of a body detection engine, or algorithm, 26 and a face detection engine, or algorithm, 28. The method may also include the step 116 of using the second video camera 14 to provide second images of the second area 20 at a second resolution that is higher than the first resolution images provided by the first video camera 12.

By analysing relatively low quality large area video images from the first video camera 12 to detect the position of the infant 22 and then targeting the detected position with the second video camera 14 to obtain high quality small area video images that are used to perform analysis to determine the predetermined infant characteristics, it is possible to reduce the amount of processing power needed by the controller and use more economic and relatively lower specification video cameras. The predetermined infant characteristics may be at least one of infant activity level, infant expression, infant happiness and infant breathing rate. These characteristics may be determined using algorithms that will be known to those skilled in the art of infant monitoring.

By way of example, a known face contour detection algorithm requires a minimum of 64×64 pixels to analyse a face and for accurate analytics normally requires at least 192×192 pixels. Assuming a face size of 0.3 m×0.3 m, this gives a requirement of 192/0.3=640 pixels/meter for accurate analytics. So, a 720p (1280×720) camera, the most common IP camera at the moment, can only provide sufficiently high resolution images for an area of 2 m×1.125 m (1280/640=1.9 meter, 720/640=1.07). To provide a high resolution image sufficient to meet the at least 192×192 pixels requirement for an area of 10.67×6 m, a camera with a resolution=6828.8 pixel×3840 pixels is required (10.67 m×640 pixel/meter=6828.8, 6 m×640 pixel/meter=3840). Using two cameras as in the infant monitoring apparatus 10, it is possible to replace a 6828×3840 pixels camera with two 720p cameras. Thus, a process that requires relatively wide area imaging for position detecting and relatively high resolution imaging for infant characteristics analysis can be achieved using video cameras not able to satisfy both requirements, while reducing the data processing demands on the device that processes the images.

The dual camera infant monitoring apparatus and methods disclosed herein may be used at least in part in combination with the breathing detection apparatus and methods for detecting breathing disclosed in the Applicant's United Kingdom Patent Application No 1914842. 8 filed on 14 Oct. 2019 and PCT Patent Application (Agent's

Reference P00649WO) filed 14 Oct. 2020, the entire content of which is incorporated herein by reference.

In the illustrated example, the dual imaging device monitoring apparatus is configured as an infant monitoring apparatus 10. It is to be understood that the monitoring apparatus is not limited to monitoring infants. Thus, for example, the monitoring apparatus may be used to monitor humans other than infants or pets. In one example, the monitoring apparatus may be used for intruder detection. For intruder detection, the images obtained by the first video camera 12 may be used to detect the position of a person in the first area 18 so that the second video camera 14 can be trained on a smaller second area 20 containing that person. The second area may contain the person's entire body or just the person's head. The higher resolution images obtained with the second video camera 14 may be compared with an images database of persons such as family members or employees that might be expected to be found in the first area 18. If the image indicates the presence of a person not to be found in an image in the images database, the controller 16 may provide an intruder alert signal that may be used to trigger an alarm signal or the sending of a message to a law enforcement or security agency address or an owner of the premises containing the first area 18. For this purpose, the controller 16 may be equipped with an alarm module configured to output an audible alarm or a telecommunications module configured to send messages to the law enforcement, security agency or owner wirelessly or via a wired network. Alternatively, the controller 16 may be coupled with known security apparatus configured to provide an alarm in response to a trigger signal or send alarm messages to at least one designated receiver.

In the illustrated example, the dual imaging device monitoring apparatus comprises first and second video cameras 12, 14. It is to be understood that the dual imaging device monitoring apparatus may be provided with imaging devices other than video cameras and is not limited to imaging devices providing chromatic imaging. For example, the monitoring apparatus may be provided with first and second imaging devices selected from one or more of the following imaging devices:
i) at least one TOF (distance) camera;
ii) at least one thermal radiation camera;
iii) at least one ultrasound camera;
iv) at least one X-ray camera;
v) at least one MRI imaging device;
vi) at least one stills camera
vii) at least one infrared camera; and
viii) at least one nuclear medicine (Gamma) camera.

Thus, in one example, the first imaging device may be a time-of-flight camera used to provide first images that are processed to locate a target object, such as a human, in the first area 18 and the second imaging device may be a high resolution stills or video camera used to provide images of the second area 20 that are analysed to determine at least one characteristic of the target object.

It is to be understood that the dual imaging device monitoring apparatus may be used to monitor target objects at indoor or outdoor locations.

1. A dual imaging device monitoring apparatus comprising:
a first imaging device to capture images of a first area;
a second imaging device to capture images of a second area that is within said first area; and
a controller connected with said first and second imaging devices and configured to analyze images received from said first imaging device to detect the position of a target object in said images, control said second video camera so that said second area includes the detected position of said target object and analyze said images from said second video camera to determine the status of at least one predetermined target object characteristic.

2. The monitoring apparatus as claimed in claim 1, wherein said second imaging device is configured to be controllable to adjust at least one of pan and tilt and said controller is configured to control at least one of said pan and tilt so that said second area contains the detected position of the target object.

3. The monitoring apparatus as claimed in claim 1, wherein said controller is configured to run at least one of a body detection engine and a face detection engine to detect said position of the target object.

4. The monitoring apparatus as claimed in claim 1, wherein said first and second imaging devices are selected from:
i) at least one video camera;
ii) at least one TOF (distance) camera;
iii) at least one thermal radiation camera;
iv) at least one X-ray camera;
v) at least one MRI imaging device;
vi) at least one ultrasound camera;
vii) at least one stills camera;
vii) at least one infrared camera; and
ix) at least one nuclear medicine camera.

5. The monitoring apparatus as claimed in claim 1, wherein said first and second imaging devices have the same imaging specification.

6. The monitoring apparatus as claimed in claim 5, wherein said first and second imaging devices are each 720p video cameras.

7. The monitoring apparatus as claimed in claim 1, wherein said target object is an infant and said at least one target object characteristic is at least one infant characteristic.

8. The monitoring apparatus as claimed in claim 7, wherein said at least one infant characteristic comprises infant activity level, infant expression, infant happiness and infant breathing rate.

9. A method of monitoring a target object comprising:
using a first imaging device to provide first images of a first area at a first resolution;
processing said first images to detect a position of said target object in said first area;
using a second imaging device to provide second images of a second area that is within said first area and contains said position at a second resolution that is higher than said first resolution.

10. The method of monitoring a target object as claimed in claim 9, further comprising causing said second imaging device to tilt, pan or tilt and pan to point said second imaging device at said position.

11. The method of monitoring a target object as claimed in claim, further comprising analyzing said second images to measure at least one target object characteristic.

12. The method of monitoring a target object as claimed in claim 11, wherein said target object is an infant and said at least one target characteristic is at least one infant characteristic.

13. The method of monitoring a target object as claimed in claim 12, wherein said at least one infant characteristic is selected from infant activity level, infant expression, infant happiness and infant breathing rate.
